(19) United States
(12) Patent Application Publication
(10) Pub. No.: US 2023/0290446 A1
(43) Pub. Date: Sep. 14, 2023

(54) FEATURIZATION OF CARBON DIOXIDE CAPTURE CHARACTERISTICS IN MOLECULES

(71) Applicant: INTERNATIONAL BUSINESS MACHINES CORPORATION, ARMONK, NY (US)

(72) Inventors: JAMES MCDONAGH, Frodsham (GB); Stamatia Zavitsanou, Oxford (GB); ALEXANDER HARRISON, Manchester (GB); FLAVIU CIPCIGAN, Warrington (GB); Dmitry Zubarev, San Jose, CA (US)

(21) Appl. No.: 17/653,897

(22) Filed: Mar. 8, 2022

(51) Int. Cl. G16C 20/62; G16C 20/64; G16C 20/70

(52) U.S. Cl. CPC G16C 20/62; G16C 20/64; G16C 20/70

(57) ABSTRACT

A method, computer program product, and computer system are provided for featurization of carbon dioxide capture characteristics in molecules. The method inputs a structure-based key of a fixed number of sub-structure descriptors for chemical groups relating to carbon dioxide capture characteristics. Sub-structure searching of each of the sub-structures defined in the key is carried out through candidate molecules represented in a format suitable for searching. A featurization of each candidate molecule is provided in the form of a fixed bit fingerprint indicating a presence or an absence of the sub-structures defined in the key. The fingerprint is applied for screening of molecules for carbon dioxide capture characteristics.

### BACKGROUND

The present invention relates to molecular identification processes, and more specifically, to featurization of carbon dioxide capture characteristics in molecules.

Carbon capture technology includes the processes involved in capturing carbon dioxide before it enters the atmosphere, transporting it, and storing it. Carbon capture technology is of huge importance due to global emphasis on environmental concerns and in particular global warming.

Carbon capture technology is a rapidly changing field with multiple technologies currently being explored. Currently, the main commercial offerings for carbon capture technology are those of solvent-based carbon capture and more specifically amine-based solvents. Absorption or carbon scrubbing with amines is the technique that is currently used industrially in which cold solutions of the amine organic compounds bind carbon dioxide.

In the field of therapeutic medicine development, high-throughput virtual screening (HTVS) methods allow scientists to rapidly perform millions of chemical, biological, pharmacological, or toxicological tests on natural compounds. Known approaches in the field of HTVS for therapeutic medicine development, use quantitative structure-activity relationship (QSAR) and quantitative structure-property relationship (QSPR) for computational modeling for revealing relationships between structural properties of chemical compounds and biological activities.

During model development, chemical descriptors are calculated on different levels of representation of molecular structure, and are then correlated with the biological property using machine learning techniques. Molecular fingerprints have been used for many years principally to enable molecular graph comparisons. Fingerprints encode the two-dimensional graph features of molecules and can be used in applications such as similarity searches, molecular characterization, molecular diversity, and chemical database clustering.

The most common forms of molecular fingerprints include circular and connectivity-based fingerprints such as Morgan fingerprints. These methods label atoms as nodes with specific properties and iteratively move n bonds away from the first atom up to some radius in the number of bonds. This provides a series of bits (n bits per atomic node) describing the molecule as a vector suitable for structural comparisons. However, as each bit may represent a different feature across molecules, such fingerprints are often not the best choice for machine learning.

Another common form of chemical fingerprints is that of fixed bit representation. In this case each bit of the fingerprint refers to a specific structure's presence, absence, or count. Molecular Access System (MACCS) fingerprints are examples of this in the form of 166 bit and 960 bit structural key descriptors in which each bit is associated with a specific molecular pattern. When a structural key is generated for a molecule, the bit string encodes whether or not these specific molecular patterns are present or absent in the molecule.

### SUMMARY

According to an aspect of the present invention there is provided a computer-implemented method for featurization of carbon dioxide capture characteristics in molecules, the method including by one or more processors of a computer system: inputting a structure-based key of a fixed number of sub-structure descriptors for chemical groups relating to carbon dioxide capture characteristics, carrying out sub-structure searching of each of the sub-structures defined in the key through candidate molecules represented in a format suitable for searching, providing a featurization of each candidate molecule in the form of a fixed bit fingerprint indicating a presence or an absence of the sub-structures defined in the key, and applying the fingerprint for screening of molecules for carbon dioxide capture characteristics.

According to another aspect of the present invention there is provided a system for featurization of carbon dioxide capture characteristics in molecules, including a processor and a memory configured to provide computer program instructions to the processor to execute methods of defined components, a key input component for inputting a structure-based key of a fixed number of sub-structure descriptors for chemical groups relating to carbon dioxide capture characteristics, a sub-graph searching component for carrying out sub-graph searching of each of the sub-structures defined in the key through candidate molecules represented in a format suitable for searching, a fingerprint component for providing a featurization of each candidate molecule in the form of a fixed bit fingerprint indicating a presence or an absence of the sub-structures defined in the key, and a fingerprint applying component for applying the fingerprint for screening of molecules for carbon dioxide capture characteristics.

According to a further aspect of the present invention there is provided a computer program product, including one or more computer readable hardware storage devices having computer readable program code stored therein, the program code containing instructions executable by one or more processors of a computer system to implement a method for featurization of carbon dioxide capture characteristics in molecules, the method including receiving an input of a structure-based key of a fixed number of sub-structure descriptors for chemical groups relating to carbon dioxide capture characteristics, carrying out sub-graph searching of each of the sub-structures defined in the key through candidate molecules represented in a format suitable for searching; providing a featurization of each candidate molecule in the form of a fixed bit fingerprint indicating a presence or an absence of the sub-structures defined in the key, and outputting the fingerprint for screening of molecules for carbon dioxide capture characteristics.

The computer readable storage medium may be a non-transitory computer readable storage medium and the computer readable program code may be executable by a processing circuit.

### BRIEF DESCRIPTION OF THE DRAWINGS

The subject matter regarded as the invention is particularly pointed out and distinctly claimed in the concluding portion of the specification. The invention, both as to organization and method of operation, together with objects, features, and advantages thereof, may best be understood by reference to the following detailed description when read with the accompanying drawings.

Embodiments of the present invention will now be described, by way of example only, with reference to the accompanying drawings:

FIG. 1 is a flow diagram of an example embodiment of a method in accordance with embodiments of the present invention;

FIG. 2 is a flow diagram of another example embodiment of a method in accordance with embodiments of the present invention;

FIG. 3 is block diagram of an example embodiment of a system in accordance with embodiments of the present invention;

FIG. 4 is a block diagram of an embodiment of a computer system or cloud server in which embodiments of the present invention may be implemented;

FIG. 5 is a schematic diagram of a cloud computing environment in which embodiments of the present invention may be implemented; and

FIG. 6 is a diagram of abstraction model layers of a cloud computing environment in which embodiments of the present invention may be implemented.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numbers may be repeated among the figures to indicate corresponding or analogous features.

### DETAILED DESCRIPTION

Embodiments of a method, system, and computer program product are provided for featurization of carbon dioxide capture characteristics in molecules.

The described method uses a defined a structure-based key of a fixed number of sub-structure descriptors for chemical groups relating to carbon dioxide capture characteristics. Sub-structure graph searching of each of the sub-structures defined in the key is carried out through candidate molecules represented in a format suitable for searching.

A featurization of the candidate molecules is provided in the form of a fixed bit fingerprint indicating a presence or an absence of the sub-structures defined in the key, with the fingerprint having potential for identifying and screening of molecules for carbon dioxide capture characteristics.

The featurization of carbon dioxide capture characteristics is an improvement in the technical field of molecular virtual screening and in the technical field of carbon capture.

There is a need to identify new carbon capture materials which have more promising characteristics than the present molecules (such as efficiency of capture). This involves searching through existing data of chemicals for promising candidates, comparing molecular structures and predicting (new and existing) properties of molecules. A method is provided to describe a molecule's structure using a computer and to apply this description to carry out the tasks described in the context of carbon capture materials discovery.

Virtual screening for molecules that have advantageous carbon capture characteristics is a valuable tool enabling expensive and time-consuming laboratory exploration to target the most promising candidates first. The current state of this field is largely laboratory-based screening, although it is envisaged that this may extend to in-field screening in the future. To enable virtual screening, molecular fingerprints are provided suitable for machine learning features and similarity searching.

Current carbon capture technologies focus on finding carbon capture amine molecules and therefore the defined structure-based key provides sub-structure descriptors for chemical groups of amine-based carbon dioxide solvents to target candidate molecules in the form of carbon dioxide capturing amine molecules. However, the described technology may be extended to define other chemical groups in the defined structure-based key as such groups that become known as relevant in the carbon capture technologies.

The described featurization presents a vector of inputs related to specific molecular rules which are fast to generate and provide physical interpretation of the featurization process. This provides an advantage over existing methods using generic featurization for these materials (for example, atom counts and charges) in one off instantiations.

The described featurization provides physically interpretable featurization in terms of the chemical moieties present and absent within a molecule. These features may be used to develop classification models for carbon capture solvents.

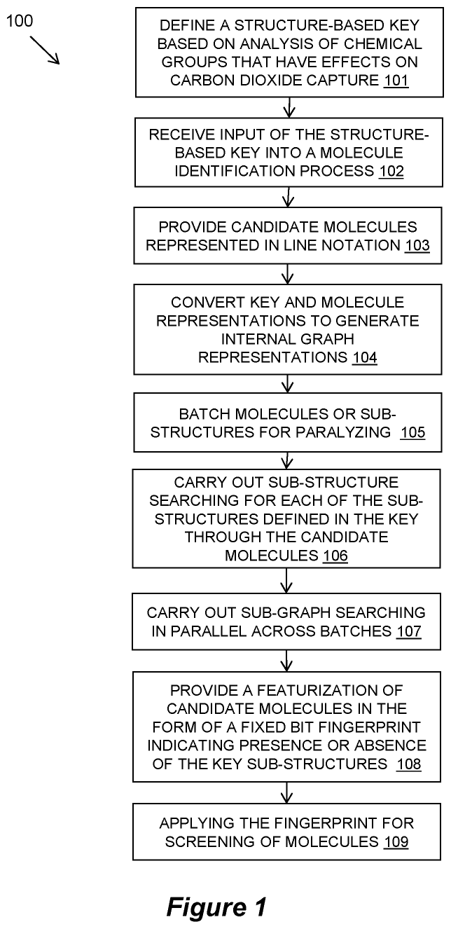

Referring to FIG. 1, a flow diagram 100 shows an example embodiment of the described method.

The method may define, 101, a structure-based key based on analysis of chemical groups that have effects on carbon dioxide capture performance to target carbon dioxide capture molecules. Chemical groups with positive characteristics for carbon capture processes may be selected and defined in the key. The chemical groups with positive characteristics may be chemical groups whose presence correlates with high carbon dioxide capture capability. In addition, chemical groups with negative characteristics for carbon capture processes may also be selected and defined in the key. The chemical groups with negative characteristics may be chemical groups underrepresented in high carbon dioxide capture materials.

In an embodiment, the chemical groups relating to carbon dioxide capture characteristics are amine-based carbon dioxide solvents to target candidate molecules in the form of carbon dioxide capturing amine molecules. The chemical groups with positive characteristics for carbon dioxide capture may focus on nitrogen containing chemical functional groups, for example, amines and heterocycles. The chemical groups with negative characteristics may be chemical groups not commonly found in carbon capture materials, for example, halogen groups and six-member aromatic carbon rings.

The method may receive input, 102, of the defined structure-based key of the fixed number of sub-structure descriptors. This may be provided in a line notation for specification of sub-structural patterns in molecules. For example, this may be simplified molecular-input line-entry system (SMILES) arbitrary target specification (SMARTS) line notation. SMILES arbitrary target specification, or SMARTS, is a language for specifying substructural patterns in molecules.

Selecting the sub-structure descriptors to define in the key is important as there are in principle for any practical purpose an infinite number that may be defined.

The method may provide, 103, candidate molecules for screening. These may be represented in a line notation. For example, this may be SMILES line notation for describing the structure of chemical species using short ASCII strings. Another example may be the International Union of Pure and Applied Chemistry (IUPAC) International Chemical Identifier (InChI) line notation, which also may be referred to as a textual identifier or as a general-purpose molecular identifier.

The method may convert, 104, the structure-based key and the candidate molecule representations to generate internal graph representations suitable for sub-structure searching for isomorphism. This may parse the structure-based key line notation and build a graph. This may also parse the candidate molecule line notation and build a graph.

The method may batch, 105, the candidate molecules or the sub-structures for parallel sub-structure searching. Knowledge of the scaling of the selected sub-structure search algorithm is used to select an appropriate batch size to parallelize over. For example, batching of 1000 molecules may provide an efficient search.

The method may carry out, 106, sub-structure searching of each of the sub-structures defined in the key through candidate molecules represented in the converted format suitable for searching. An efficient search is carried out of for each sub-structure in the molecule graph. The order of inputs and outputs is tracked to make the correct representation match up.

The sub-structure searching may be parallelized, 107, using a parallel computing library. The sub-structure search parallel processing must maintain the order such that the output matches the order of the input. The sub-structure searching may be parallelized by batching whilst maintaining the order. The batching may batch the candidate molecules for parallel searching. Alternatively, the sub-structure searching may be parallelized over the sub-structures.

The method may provide, 108, a featurization of the candidate molecules in the form of a fixed bit fingerprint indicating a presence or an absence of the sub-structures defined in the key. The fingerprint describes each candidate molecule in terms of a presence or an absence. The fingerprint also describes a number of the chemical groups which have an effect on the candidate molecule's ability to capture carbon dioxide. This effect may be a positive or a negative effect and the absence or presence may be interpreted accordingly. The fingerprint may provide a bit vector of 1 or 0 value for each bit representing the presence and absence respectively of a given sub-structure of the key.

The method may provide the fingerprint for applying, 109, the fingerprint for screening of molecules for carbon dioxide capture characteristics. This application may include developing models for carbon dioxide capturing molecules, including classification models, regression models, and ranking models. The application may include running machine learning models using the fingerprint to predict molecule properties related to carbon dioxide capture uses. Methods may run natively on the cloud to featurize molecules and run machine learning models to predict molecule properties related to carbon capture uses.

Applying, 109, the fingerprint may include using the fingerprint to filter and search molecules in a database. As a database is populated, the structure-based fingerprint may be included in the entry such that the molecules can be filtered and searched based on the defined sub-structures of the fingerprint. For example, a filter may be applied for all molecules which contain a benzene ring and the search process simply is bit X=1. This is straightforward and non-computationally taxing compared to a sub-structure search.

The described method includes the generation of a chemical fingerprint representation specifically for carbon capture solvents, and the cloud-based deployment of codes capable of generating this representation efficiently.

The method may be deployed in a cloud environment to featurize candidate molecules. The method may be deployed directly on cloud-based hub instances and within computational workflows using a workflow engine to drive calculation directly on the cloud environment. Cloud based workflow applying these tools enables a rapid generation of these fingerprints over large datasets in a reproducible manner.

The computationally tractable means for the generation of the representations and a cloud-based platform to serve them allows them to be used as a component within larger accelerated discovery workflows.

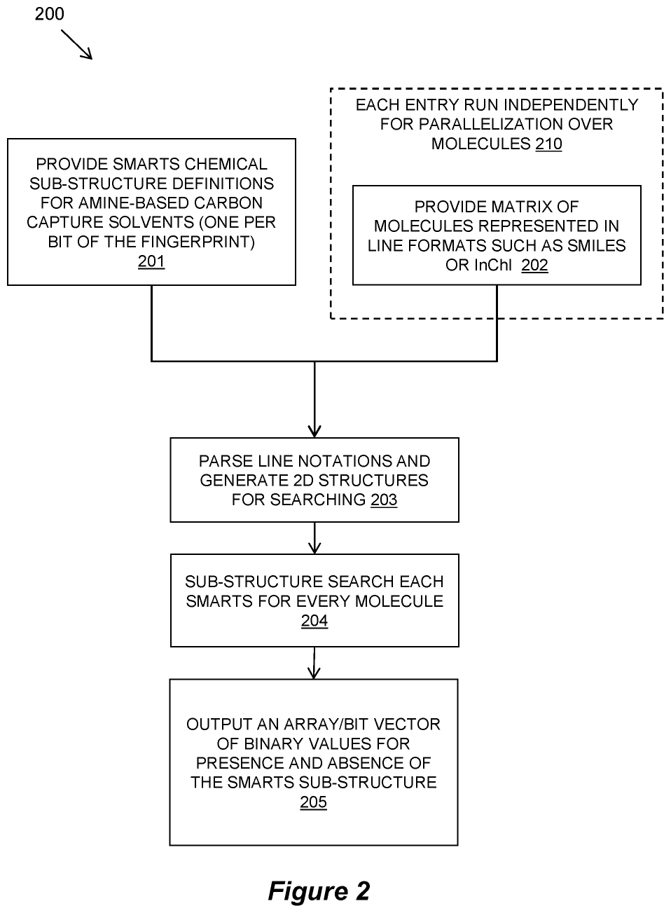

Referring to FIG. 2, a flow diagram 200 shows another example embodiment of the described method in the context of screening for amine-based solvents relevant for carbon dioxide capture. The fingerprint that is used for the featurization of molecules is broadly applicable across the amine-based molecules. Presently amine molecules are the key technology in most carbon capture settings.

SMARTS chemical sub-structure definitions are provided, 201, for amine-based carbon capture solvents as a structure-based key with one definition per bit of the fingerprint. The specific choices for the keys are unique and encode a distillation of chemical knowledge regarding the properties of amine-based solvents relevant for carbon dioxide capture. The keys are chosen on the basis of their utility for the classification of amine-based carbon dioxide solvents

The use of the fixed bit representation as a fingerprint for featurization means that the aim is to describe a molecule in terms of the presence, absence and number of chemical groups which appear to have an effect on a molecules ability to capture carbon dioxide. An example fingerprint is 64 bits long and seeks to include chemical groups whose presence correlates with high carbon capture capacity. Bits are also included in the fingerprint which correspond to common structures in synthetically available commercial amine molecules that are underrepresented in the area of carbon capture solvents. The inclusion of the latter enables stronger differentiation between the likelihood of good carbon capture capabilities.

A matrix of molecules is provided, 202, represented in a line formation, for example, SMILES or InChI. In this embodiment, parallelization, 210, is provided for molecules enabling each entry to be run independently. This process is built into a cloud-based workflow for easy deployment and transferability. The parallelized implementation calculates the fingerprints using standard programming packages (for example, Python packages) leads to a ˜260× speed up compared to serial processing over ˜12,000 molecules. Python is a trademark of Python Software Foundation.

The line notations are parsed and two-dimensional structures are generated, 203, for the sub-structure definitions of the structure-based key and for the molecules. Sub-structure searching, 204, is carried out for each SMARTS of the key for every molecule using the parallelization 210.

As sub-structure searching is a computationally demanding task, for high throughput screening, the process is parallelized. Each bit in the fingerprint corresponds to a given sub-structure each of which is defined using chemical SMARTS notation. Cheminformatics software (for example, the open-source package RDKit) provides a sub-structure searching routine which is applied to build and output, 205, a bit vector of 1 or 0 value for each bit representing presence and absence of a given sub-structure. This process is parallelized by batching the calculation into groups of 1000 molecules and using a parallel computing library (for example, DASK library in Python) to execute all sub-structure searches in parallel. RDKit is a trademark of T5 Informatics GmbH.

The implementation has been tested over a data set of 11,725 molecules running natively using Python libraries. Parallelizing as described reduced the execution time over 11,725 molecules from 11 hours 48 minutes in the serial routine to 2 minutes 42 seconds. The methods may be built such that they are callable and executable from a single command run through scripts and notebooks. The method may be run via a hub installed on a cloud infrastructure. These methods may also be built into a workflow which is directly run on cloud instances.

The featurization provided by the fingerprint potentially makes machine learning tasks relating to carbon capture more sample efficient. The method provides physically interpretable featurization in terms of the chemical moieties present and absent within a molecule. These features may be used to develop classification models, regression models, and ranking models for carbon capture solvents. There models provide a similar level of accuracy to the use of traditional cheminformatics descriptors but additionally enable similarity and structural analysis within the context of carbon capture materials, all from one representation.

These models provide good discriminatory behavior between good carbon dioxide absorbing molecules and poor carbon dioxide absorbing molecules. Recent tests have shown 84% of a dataset was classified correctly using the fingerprints as features for machine learning classifiers. Additionally, the fingerprints enable similarity measurement and structural analysis within the context of carbon capture materials all from one representation.

The combination of sub-structures decisions which are extracted based on analysis and published data has led to a fingerprint targeting carbon capture molecules. This capability is extended by parallelizing the algorithm to avoid high computation costs from many sub-structure searches.

The code may be provided into a platform which can be automatically deployed onto cloud clusters for efficient computation.

TABLE 1
Example definitions that may be included in the structure-based
key for the fingerprint are shown in the table.

| Chemical Group | SMARTS notation |
|---|---|
| primary amine | [NX3; H2; !$(NC═O)][#6X4] |
| secondary amine | [NX3; H1; !$(NC═O)]([#6X4])[#6X4] |
| tertiary amine | [NX3; !$(NC═O)]([#6X4])([#6X4])[#6X4] |
| primary alcohol | [#6][CH2][OH] |
| secondary alcohol | [#6][CH]([#6])[OH] |
| six-membered | c1ccccc1 |
| aromatic carbon | |
| ring | |

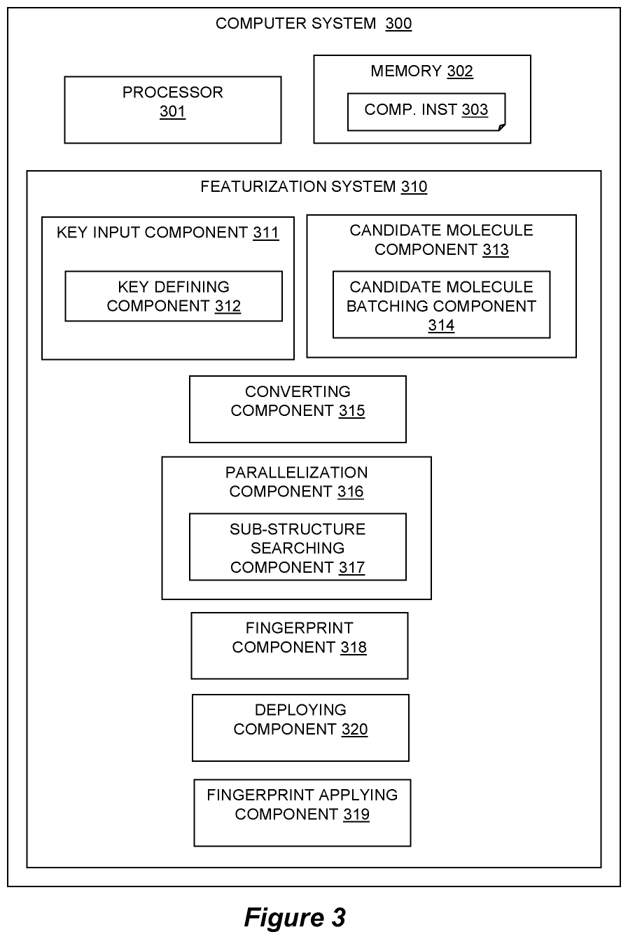

Referring to FIG. 3, a block diagram shows an example embodiment of a computing system 300 in which a featurization system 310 for featurization of carbon dioxide capture characteristics in molecules may be provided.

The computing system 300 may include at least one processor 301, a hardware module, or a circuit for executing the functions of the described components which may be software units executing on the at least one processor. Multiple processors running parallel processing threads may be provided enabling parallel processing of some or all of the functions of the components. Memory 302 may be configured to provide computer instructions 303 to the at least one processor 301 to carry out the functionality of the described components.

The featurization system 310 may include a key input component 311 for inputting a structure-based key of a fixed number of sub-structure descriptors for chemical groups relating to carbon dioxide capture characteristics. The key input component 311 may include a key defining component 312 for defining the structure-based key based on analysis of chemical groups that have effects on carbon dioxide capture performance to target carbon dioxide capture molecules.

The featurization system 310 may include a candidate molecule component 313 for providing candidate molecules represented in line notation. The candidate molecule component 313 may include a candidate molecule batching component 314 for batching the candidate molecules for parallel processing.

The featurization system 310 may include a converting component 315 for converting the key and candidate molecule representations to generate internal graph representations suitable for sub-structure searching. The structure-based key is provided in a line notation for specification of sub-structural patterns, and the target molecules are provided in a line notation and the converting component 315 generates internal graph representations from the line notations for sub-structure searching.

The featurization system 310 may include a sub-structure search component 317 for carrying out sub-structure searching of each of the sub-structures defined in the key through candidate molecules represented in a format suitable for searching. The sub-structure search component 317 may be parallelized by a parallelization component 316 providing multiple instances of the sub-structure search component 317 and using parallel computing libraries.

The featurization system 310 may include a fingerprint component 318 providing a featurization of the candidate molecules in the form of a fixed bit fingerprint indicating a presence or an absence of the sub-structures defined in the key. The resultant fingerprint describes each candidate molecule in terms of a presence, an absence, and a number of the chemical groups which have an effect on the candidate molecule's ability to capture carbon dioxide.

The featurization system 310 may include a deploying component 320 for deploying the featurization system 310 or components thereof in a cloud environment to featurize candidate molecules. The featurization system 310 may be deployed directly on cloud-based hub instances and within computational workflows using a workflow engine to drive calculation directly on the cloud environment in a reproducible manner.

The featurization system 310 may include a fingerprint applying component 319 for applying the resultant fingerprint for screening of molecules for carbon dioxide capture characteristics. The fingerprint applying component 319 may output the fingerprint results to external processes. The external processing may include developing classification models, regression models and ranking models for carbon dioxide capturing molecules. The external processing may include running machine learning models using the fingerprint to predict molecule properties related to carbon dioxide capture uses.

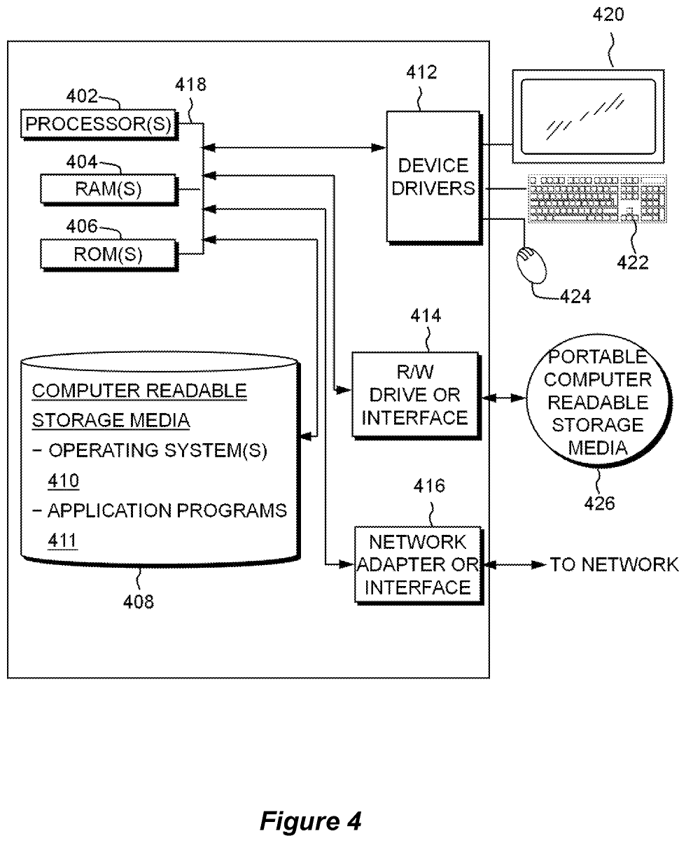

FIG. 4 depicts a block diagram of components of a computing system as used for the computing system 300 of FIG. 3, in accordance with an embodiment of the present invention. It should be appreciated that FIG. 4 provides only an illustration of one implementation and does not imply any limitations with regard to the environments in which different embodiments may be implemented. Many modifications to the depicted environment may be made.

The computing system can include one or more processors 402, one or more computer-readable RAMs 404, one or more computer-readable ROMs 406, one or more computer readable storage media 408, device drivers 412, read/write drive or interface 414, and network adapter or interface 416, all interconnected over a communications fabric 418. Communications fabric 418 can be implemented with any architecture designed for passing data and/or control information between processors (such as microprocessors, communications and network processors, etc.), system memory, peripheral devices, and any other hardware components within the system.

One or more operating systems 410, and application programs 411, such as the featurization system components, are stored on one or more of the computer readable storage media 408 for execution by one or more of the processors 402 via one or more of the respective RAMs 404 (which typically include cache memory). In the illustrated embodiment, each of the computer readable storage media 408 can be a magnetic disk storage device of an internal hard drive, CD-ROM, DVD, memory stick, magnetic tape, magnetic disk, optical disk, a semiconductor storage device such as RAM, ROM, EPROM, flash memory, or any other computer readable storage media that can store a computer program and digital information, in accordance with embodiments of the invention.

The computing system can also include the R/W drive or interface 414 to read from and write to one or more portable computer readable storage media 426. Application programs 411 on the computing system can be stored on one or more of the portable computer readable storage media 426, read via the respective RAY drive or interface 414 and loaded into the respective computer readable storage media 408.

The computing system can also include the network adapter or interface 416, such as a TCP/IP adapter card or wireless communication adapter. Application programs 411 on the computing system can be downloaded to the computing device from an external computer or external storage device via a network (for example, the Internet, a local area network or other wide area networks or wireless networks) and network adapter or interface 416. From the network adapter or interface 416, the programs may be loaded into the computer readable storage media 408. The network may comprise copper wires, optical fibers, wireless transmission, routers, firewalls, switches, gateway computers and edge servers.

The computing system can also include a display screen 420, a keyboard or keypad 422, and a computer mouse or touchpad 424. Device drivers 412 interface to display screen 420 for imaging, to keyboard or keypad 422, to computer mouse or touchpad 424, and/or to display screen 420 for pressure sensing of alphanumeric character entry and user selections. The device drivers 412, R/W drive or interface 414, and network adapter or interface 416 can comprise hardware and software stored in computer readable storage media 408 and/or ROM 406.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access a normalized search engine or related data available in the cloud. For example, the normalized search engine could execute on a computing system in the cloud and execute normalized searches. In such a case, the normalized search engine could normalize a corpus of information and store an index of the normalizations at a storage location in the cloud. Doing so allows a user to access this information from any computing system attached to a network connected to the cloud (e.g., the Internet).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

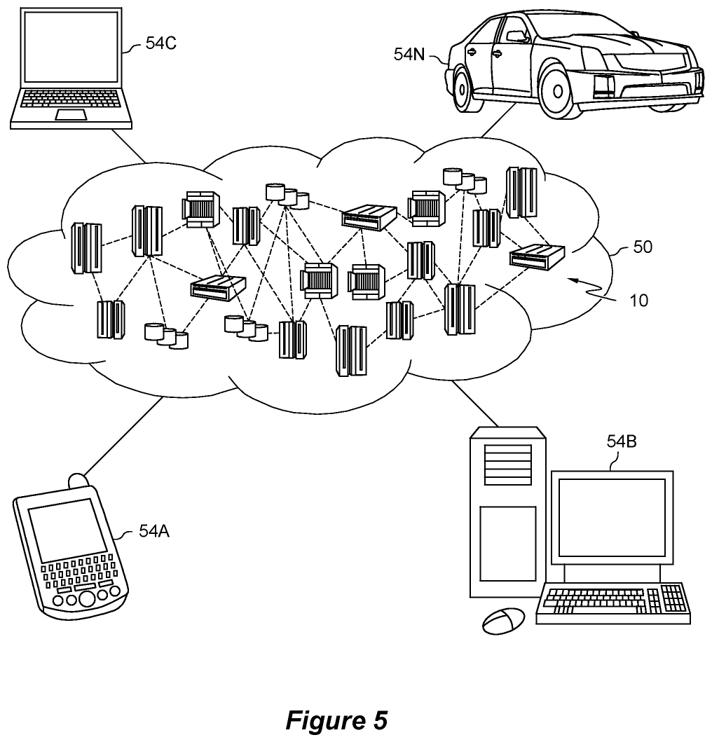

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

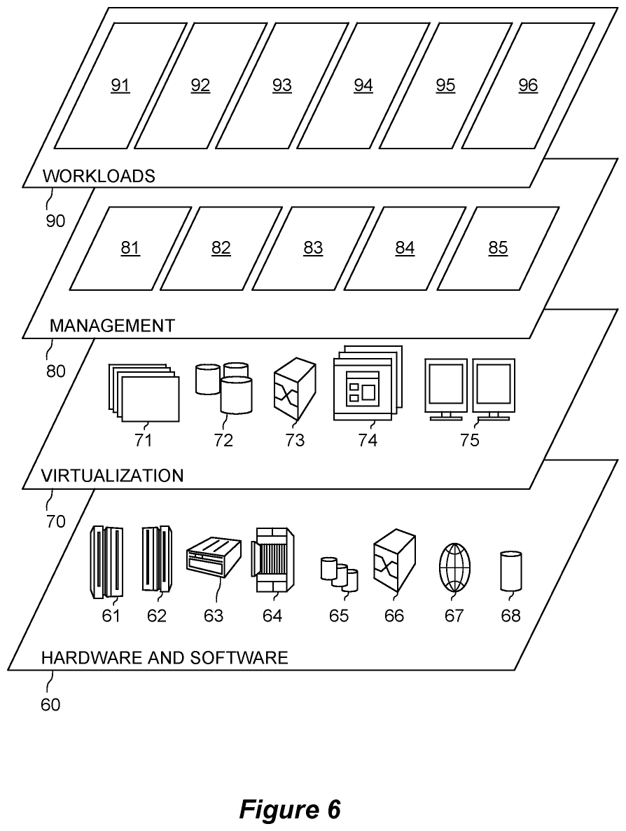

Referring now to FIG. 6, a set of functional abstraction layers provided by cloud computing environment 50 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and featurization processing 96.

A computer program product of the present invention comprises one or more computer readable hardware storage devices having computer readable program code stored therein, the program code executable by one or more processors to implement the methods of the present invention.

A computer system of the present invention comprises one or more processors, one or more memories, and one or more computer readable hardware storage devices, the one or more hardware storage device containing program code executable by the one or more processors via the one or more memories to implement the methods of the present invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Improvements and modifications can be made to the foregoing without departing from the scope of the present invention.

What is claimed is:

1. A computer-implemented method for featurization of carbon dioxide capture characteristics in molecules, the method comprising:
inputting a structure-based key of a fixed number of sub-structure descriptors for chemical groups relating to carbon dioxide capture characteristics;
carrying out sub-structure searching of each of the sub-structures defined in the key through candidate molecules represented in a format suitable for searching;
providing a featurization of each candidate molecule in the form of a fixed bit fingerprint indicating a presence or an absence of the sub-structures defined in the key; and
applying the fingerprint for screening of molecules for carbon dioxide capture characteristics.

2. The method of claim 1, wherein
carrying out sub-structure searching is parallelized by batching and executing in parallel using a parallel computing library.

3. The method of claim 1, wherein
the structure-based key is provided in a line notation for specification of sub-structural patterns, and the candidate molecules are provided in a line notation, wherein the method includes parsing the line notations to generate internal graph representations for sub-structure searching.

4. The method of claim 1, wherein the chemical groups relating to carbon dioxide capture characteristics include one or both of: chemical groups whose presence correlates with high carbon dioxide capture capability; and
chemical groups underrepresented in high carbon dioxide capture materials.

5. The method of claim 1, wherein
the fingerprint describes each candidate molecule in terms of a presence, an absence, and a number of the chemical groups which have an effect on the candidate molecule's ability to capture carbon dioxide.

6. The method of claim 1, further comprising:
defining the structure-based key based on analysis of chemical groups that have effects on carbon dioxide capture performance to target carbon dioxide capture molecules.

7. The method of claim 1, wherein
the chemical groups relating to carbon dioxide capture characteristics are amine-based carbon dioxide solvents to target candidate molecules in the form of carbon dioxide capturing amine molecules.

8. The method of claim 1, wherein
applying the fingerprint for screening of molecules for carbon dioxide capture characteristics comprises developing one or more of the group of: classification models, regression models, and ranking models for carbon dioxide capturing molecules.

9. The method of claim 1, wherein
applying the fingerprint for screening of molecules for carbon dioxide capture characteristics comprises running machine learning models using the fingerprint to predict molecule properties related to carbon dioxide capture uses.

10. The method of claim 1, further comprising:
deploying the method in a cloud environment to featurize candidate molecules comprising deployed directly on cloud-based hub instances and within computational workflows using a workflow engine to drive calculation directly on the cloud environment in a reproducible manner.

11. The method as claimed in claim 1, wherein
applying the fingerprint for screening of molecules for carbon dioxide capture characteristics comprises providing an entry in a molecule database for the fingerprint for filtering and searching based on the sub-structures of the fingerprint.

12. A computer system for featurization of carbon dioxide capture characteristics in molecules, the computer system comprising:
one or more computer processors, one or more computer-readable storage media, and program instructions stored on the one or more of the computer-readable storage media for execution by at least one of the one or more processors;
a processor and a memory configured to provide computer program instructions to the processor to execute methods of defined components;
a key input component for inputting a structure-based key of a fixed number of sub-structure descriptors for chemical groups relating to carbon dioxide capture characteristics;
a sub-structure searching component for carrying out sub-structure searching of each of the sub-structures defined in the key through candidate molecules represented in a format suitable for searching;
a fingerprint component for providing a featurization of each candidate molecule in the form of a fixed bit fingerprint indicating a presence or an absence of the sub-structures defined in the key; and
a fingerprint applying component for applying the fingerprint for screening of molecules for carbon dioxide capture characteristics.

13. The computer system of claim 12, wherein
the sub-structure searching component is parallelized by batching and executing the sub-structure searches in parallel using a parallel computing library.

14. The computer system of claim 12, further comprising:
a converting component to convert the structure-based key provided in a line notation for specification of sub-structural patterns, and the candidate molecules provided in a line notation to generate internal graph representations for sub-structure searching.

15. The computer system of claim 12, further comprising:
a key defining component for defining the structure-based key based on analysis of chemical groups that have effects on carbon dioxide capture performance to target carbon dioxide capture molecules.

16. The computer system of claim 12, wherein
the fingerprint applying component comprises developing classification models, regression models, and/or ranking models for carbon dioxide capturing molecules.

17. The computer system of claim 12, wherein
the fingerprint applying component comprises running machine learning models using the fingerprint to predict molecule properties related to carbon dioxide capture uses.

18. The computer system of claim 12, wherein
the system is deployed directly on cloud-based hub instances and within computational workflows using a workflow engine to drive calculation directly on a cloud environment in a reproducible manner.

19. The computer system of claim 12, further comprising:
a molecule database comprising an entry for the fingerprint for filtering and searching based on the sub-structures of the fingerprint.

20. A computer program product for featurization of carbon dioxide capture characteristics in molecules, the computer program product comprising:
one or more computer-readable storage media and program instructions stored on the one or more computer-readable storage media, the program instructions executable by a computing system to cause the computing system to perform a method comprising:
receiving an input of a structure-based key of a fixed number of sub-structure descriptors for chemical groups relating to carbon dioxide capture characteristics;
carrying out sub-structure searching of each of the sub-structures defined in the key through candidate molecules represented in a format suitable for searching;
providing a featurization of each candidate molecule in the form of a fixed bit fingerprint indicating a presence or an absence of the sub-structures defined in the key; and
outputting the fingerprint for screening of molecules for carbon dioxide capture characteristics.
